(19) United States
(12) Patent Application Publication
(10) Pub. No.: US 2002/0038020 A1
(43) Pub. Date: Mar. 28, 2002

(54) Process for extracting glycoside using an aqueous two-phase system

(76) Inventors: I-Horng Pan, Hsinchu (TW); Lain-Tze Lee, Hsinchu (TW); His-Ho Chiu, Hsinchu (TW); Huei-Ju Liu, Nantou (TW); Hsin-Jan Yao, Yunlin (TW)

Correspondence Address: RABIN & CHAMPAGNE, PC, 1101 14TH STREET, NW, SUITE 500, WASHINGTON, DC, 20005, US

(21) Appl. No.: 09/888,627

(22) Filed: Jun. 26, 2001

(30) Foreign Application Priority Data
Sep. 25, 2000 (TW) 89119776

(51) Int. Cl. C07H 1/06

(52) U.S. Cl. 536/128

(57) ABSTRACT

An aqueous two-phase system for extracting glycosides from a herb includes the extractive solution, about 5 wt %-30 wt % of salt, about 5 wt %-30 wt % of polyol and about 0.5 wt %-30 wt % of alcohols. The salt can be dihydrogen phosphate, hydrogen phosphate and phosphate or a mixture thereof, and it also can be sulfates, chlorides, oxalates, or acetates. In addition, a process for extracting glycosides using an aqueous two-phase system is disclosed. The procedures includes preparation of extractive solution, concentration and drying, phase separation, concentration of the water layer and drying, washing with solvent, sonication, centrifugation, concentration of filtrate and drying of the final products.

### BACKGROUND OF THE INVENTION

This application incorporates by reference Taiwanese application Serial No. 89119776, Filed Sep. 25, 2000.

1. Field of the Invention

The invention relates in general to a technique of extraction, and more particularly to the extracting glycosides from a herb using an aqueous two-phase system.

2. Description of the Related Art

With the rapid development of biotechnology, the techniques of separating and extracting biologically important compounds from animal and plant sources have become an area of great importance. The problems of traditional separation technologies include low recovering yields, and, especially, low yield resulting from fermentation of the solution due to the long period of extraction. These problems hinder the development of medication to a certain extent. The traditional process of separation and purification depend on the chemical and physical properties of materials and products, however, the method mainly applied is that of chromatography. There are several drawbacks to the use of traditional solvents and chromatography including:

1. The process of extraction is time-consuming.

2. Solvents such as dichloromethane or chloroform applied in the process of extraction are toxic and hazardous to human, as well as cause serious environmental pollution.

3. The cost of the process is high.

4. Undesired chemical reactions occur during purification.

### SUMMARY OF THE INVENTION

The objective of this invention, therefore, is to develop a method of phase separation using an aqueous two-phase system that possess advantages such as simple procedures and equipments, low-cost production, and partial recycling and reuse of materials used in the process.

According to the objective of present invention, a process for the extraction of glycosides from herb described as follows:

(a) A herb is ground and mixed with water and then agitated. After filtration, the first filtrate is collected in a beaker while the residue is removed and mixed with water and filtered again. An aqueous extractive solution is made up of the first and the second crude extracts.

(b) The aqueous extractive solution is concentrated to about 1-10% of solid content.

(c) 5 wt %˜30 wt % of salt, 5 wt %˜30 wt % of polyol and 0.5 wt %˜30 wt % of alcohol are added to the concentrated solution and mixed thoroughly. Phase separation is then carried out at a temperature between 4° C. to 90° C., after which the aqueous layer is separated from the polyol layer. The preferred temperature is in the range of room temperature—70° C.

(d) The aqueous layer is removed from the two-phase system, and concentrated in vacuo to give a solid which is dried.

(e) The resulting solids in step (d) is then suspended with a solvent to a concentration of 60%˜99%, and then sonicated. The first filtrate and residue are then obtained after centrifugation and filtration.

(f) Repeat step (e), to obtain the second filtrate and residue.

(g) The first and second filtrates are combined and concentrated in vacuo to give the final solid product rich in glycosides.

The invention achieves the extraction of glycosides from a herb by providing a new aqueous two-phase system separation method that includes an aqueous extractive solution, 5 wt %˜30 wt % of salt, 5 wt %˜30 wt % of polyol, and 0.5 wt %˜30 wt % of alcohol. The salts can be dihydrogen phosphate, hydrogen phosphate, phosphate, or a mixture thereof, it also can be sulfates, chlorides, oxalates, or acetates.

### BRIEF DESCRIPTION OF THE DRAWINGS

Other objects, features, and advantages of the invention will become apparent from the following detailed description of the preferred but non-limiting embodiments. The description is made with reference to the accompanying drawings in which:

FIG. 1 shows the procedures of extracting glycosides from a plant such as a herb using an aqueous two-phase system

### DESCRIPTION OF THE PREFERRED EMBODIMENT

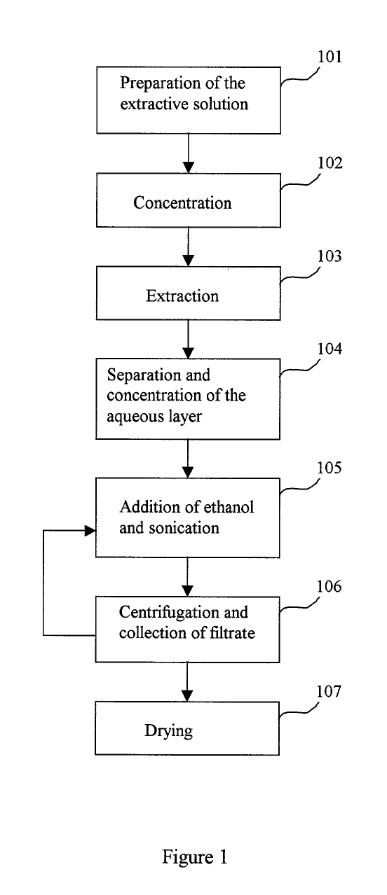

The process of extraction of glycosides from plants such as herbs using an aqueous two-phase system is presented in FIG. 1. In step 101 (extractive solution), the extractive solution is prepared by addition of crushed herb or plant to a suitable amount of water such that the herb is covered completely by the water. The extraction can be improved by agitating the mixture. The mixture is filtered and the residue is placed into water again; this step could be repeated several times. Whether the solution is heated or boiled depend on the properties of material (herbs or plants) such as difficulty of extraction and sensitivity of temperature.

The next step (102) is concentration, as shown in FIG. 1. The extractive solution is preferably concentrated to 1-10% of solid content. Whether this step is performed depend on the situation of extraction; sometimes it is not necessary to concentrate the extractive solution.

The invention of extraction using an aqueous two-phase system is disclosed in step 103. The aqueous two-phase system is preferably carried out by using an aqueous extraction solution, 5 wt %˜30 wt % of salts, and 5 wt %˜30 wt % of polyol such as PE62 (Copolymer (20/80) of ethylene oxide and propylene oxide). The salts can be dihydrogen phosphate, hydrogen phosphate, phosphate, or a mixture thereof; it also can be sulfates, chlorides, oxalates, or acetates. The aqueous two-phase system is obtained by the addition of salts and polyol to the concentrated extractive solution (prepared in step 2) or extractive solution (prepared in step 1), and then mixed thoroughly. The phase separation is carried out at a temperature between 4° C. to 90° C., with a preferred temperature range of 25° C. (room temperature)—70° C. It is more preferable to treat it with water bath, thereby improving the separation of water layer and polyol layer. In addition, the aqueous two-phase system is preferably generated by addition of alcohols (C₁-C₄) such as 0.5 wt %˜30 wt % of ethanol.

In step 104, the aqueous layer is removed from the two-phase system, preferably performed by centrifugation and filtration, and concentrated in vacuo to give a solid which is dried.

Then, the solid is washed with a solvent (60%˜99%) such as ethanol, as shown in step 105. After sonication, the solution is centrifuged and filtered to separate the filtrate and residue, as shown in step 106, and residue is removed and washed with solvent again. Steps 105 and 106 can be repeated in order to obtain glycosides from the solids adequately. All of the filtrate collected from step 106 is preferably concentrated in vacuo to obtain a final solid product rich in glycosides.

According to the preferred embodiment of the invention, monosaccharide, disacchride and polysacchride, such as sennoside, geniposide, paeoniflorin, glycyrrhizin, guercitrin, puerarin, hesperidin, ginsenoside Rbl and naringin, are extracted efficiently from herbs using the aqueous two-phase system.

According to the preferred embodiment of the invention, described above, the advantages of extracting glycosides using an aqueous two-phases system include:

1. The process is simplified due to simple procedures.

2. Extraction using aqueous two-phase system doesn't cause pollution as traditional method of extraction using organic solvent does. Part of material also can be recycled and reused in the process.

3. Simple equipments and procedures decrease the cost of production.

The following examples illustrate this invention: extraction of glycosides from dried gardenia fruit, rhubarb, and root of herbaceous peony using an aqueous two-phase system.

### Example 1

Extracting glycosides from dried gardenia fruit using an aqueous two-phase system.

1. 5 g of dried gardenia fruit was mildly ground, mixed with 100 g of water, and boiled for 30 minutes. After filtration, the filtrate was collected and the remaining gardenia fruit was mixed with fresh water, boiled, and filtered. The aqueous extractive solution was obtained by combining the two filtrates. This extractive solution contained 26.93 wt % of geniposide as determined via HPLC.

2. 155.59 g of the extractive solution is concentrated to 21.79 g. The solid content of the extractive solution concentrate is approximately 8.23%.

3. 1.64 g (7.5%) of potassium dihydrogen phosphate (KH₂PO₄), 1.09 g (5%) of PE62 and 2 ml (10%) of ethanol are added to the extractive solution concentrate and then mixed thoroughly. The phase separation is performed by keeping the solution in a water bath at a temperature of 70° C. for 1-1.5 hour.

4. After phase separation, the upper layer is the aqueous while the lower layer was PE62. The aqueous layer is carefully removed and concentrated in vacuo to give a solid which is dried.

5. 5-10 ml of 95% ethanol is added to dried solid, and the mixture is sonicated for 3 minutes.

6. Subsequently, the mixtures are centrifuged and the solid remaining is collected in a beaker. 5-10 ml of 95% ethanol was added to the residue (solids), followed by sonicating for 3 minutes.

7. Thereafter, the filtrate was separated from the residue by centrifugation. Concentration of the filtrate in vacuo gave a solid which is dried. The weight of solid product was 0.79 g and the geniposide was 318.44 mg determined by means of HPLC. The ratio of geniposide was 40.31%.

In this case, the geniposide of dried gardenia fruit is increased from 26.93% to 40.31% using the aqueous two-phases system. This process improves yields of the glycosides.

### Example 2

Extracting glycosides from rhubarb using an aqueous two-phase system.

1. 10 g of rhubarb was mildly ground and mixed with 300 g of water. the mixture was agitated (200 rpm) at room temperature for 30 minutes. After filtration, the filtrate was collected as the aqueous extractive solution. This extractive solution contained 3.71% of sennosides as determined by HPLC.

2. 115 ml of the extractive solution is concentrated to 20 ml. The solid content of the extractive solution concentrate is approximately 6.43%.

3. 1 g (5%) of sodium biphosphate (Na₂HPO₄), 2 g (10%) of PE62 and 4 ml (20%) of ethanol are added to the extractive solution concentrate and then mixed thoroughly. The phase separation is performed by keeping the solution in a water bath at a temperature of 70° C. for 1-1.5 hour.

4. The upper layer (aqueous) is separated from the lower layer (PE62) using centrifugation (3000 rpm). Then, the water layer is carefully removed and concentrated in vacuo to a dry solid.

5. 5-10 ml of 70% ethanol is added to dried solid, and the solution was sonicated for 3 minutes.

6. Subsequently, the phases are separated by centrifugating and the filtrate is collected in a beaker. 5-10 ml of 75% ethanol is added to the residue (solids), and the mixture is sonicated for 3 minutes.

7. Thereafter, the filtrate is separated from residue by centrifugation. The combined filtrate is concentrated in vacuo to dryness. The weight of solid product is 0.40 g and the sennosides is 24.2 mg as determined by HPLC. The ratio of sennosides is 6.05%.

In this case, the sennosides of rhubarb is increased from 3.71% to 6.05% using aqueous two-phases system. This process improves yields of the glycosides.

### Example 3

Extracting glycosides from root of herbaceous peony using an aqueous two-phase system.

1. 10 g of root of herbaceous peony was mildly ground, mixed with 200 g of water, and boiled for 60 minutes. After filtration, the filtrate was collected and the remaining root of herbaceous peony was again mixed with water, boiled, and filtered. The aqueous extractive solution was obtained by combining the filtrates. This extractive solution contained 1.36% of paeoniflorin as determined by HPLC.

2. 158 g of the extractive solution is concentrated to 40.65 g. The solid content of the extractive solution concentrate is approximately 4.52%.

3. 3.2 g (8%) of sodium dihydrogen phosphate (NaH₂PO₄), 4.8 g (12%) of PE62 and 4 ml (10%) of ethanol are added to the extractive solution concentrate and then mixed thoroughly. The phase separation is performed by keeping the solution in water bath at a temperature of 70° C. for 1-1.5 hour.

4. The upper layer (aqueous) was carefully removed from the lower layer (PE62) and concentrated in vacuo to a dry solid.

5. 5-20 ml of 95% ethanol is added to the dried solid, and the solution was sonicated for 30 minutes.

6. Subsequently, the phases are separated by centrifugation and the filtrate is collected in a beaker. 5-20 ml of 95% ethanol was added to the residue followed by sonicating for 30 minutes. The mixture was kept at room temperature overnight.

7. Thereafter, the filtrate was separated from residue by centrifugation. The combined filtrates were concentrated in vacuo to dryness. The weight of solid product was 0.69 g and the paeoniflorin was 13.8 mg as determined by HPLC. The ratio of paeoniflorin was 2%.

In this case, the paeoniflorin of root of herbaceous peony is increased from 1.36% to 2% using aqueous two-phases system. This process improves yields of the glycosides.

While the invention has been described by way of example and in terms of the preferred embodiment, it is to be understood that the invention is not limited to the disclosed embodiment. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A process of extracting glycosides from a plant, comprising the steps of:
(a) grinding the plant and mixing with water to form an extractive solution;
(b) adding about 5 wt %˜30 wt % of salt and about 5 wt %˜30 wt % of polyol to the extractive solution and then mixing thoroughly to form an aqueous layer and a polyol layer;
(c) removing the aqueous layer and concentrating the aqueous layer to be a solid;
(d) washing the solid with a solvent, and producing a filtrate and collecting the filtrate after filtration; and
(e) concentrating the filtrate to obtain a final product rich in glycosides.

2. The process of extracting glycosides according to claim 1, wherein the plant is a herb.

3. The process of extracting glycosides according to claim 2, wherein the herb is root of herbaceous peony.

4. The process of extracting glycosides according to claim 2, wherein the herb is dried gardenia fruit.

5. The process of extracting glycosides according to claim 2, wherein the herb is rhubarb.

6. The process of extracting glycosides according to claim 1, wherein the glycosides include monosaccharide, disacchrides or polysacchride.

7. The process of extracting glycosides according to claim 6, wherein the glycoside consist of sennoside, geniposide, paeoniflorin, glycyrrhizin, guercitrin, puerarin, hesperidin, ginsenoside Rbl and naringin.

8. The process of extracting glycosides according to claim 1, wherein the step (a) further comprises the steps of boiling and agitation.

9. The process of extracting glycosides according to claim 1, wherein the step (a) further comprises the steps of adding water, boiling, and agitating for several times to form a crude extract for each time and each of the crude extract is collected and combined to form the extractive solution.

10. The process of extracting glycosides according to claim 1, wherein after the step (a) and before the step (b), a concentration procedure is further performed to concentrate the extractive solution to form an extractive solution concentrate with a solid content of 1-10%.

11. The process of extracting glycosides according to claim 1, wherein a salt is selected from the group consisting of dihydrogen phosphate, hydrogen phosphate and phosphate.

12. The process of extracting glycosides according to claim 1, wherein the salt in step (b) is sulfate.

13. The process of extracting glycosides according to claim 1, wherein the salt in step (b) is chloride.

14. The process of extracting glycosides according to claim 1, wherein the salt in step (b) is oxalate.

15. The process of extracting glycosides according to claim 1, wherein the salt in step (b) is acetate.

16. The process of extracting glycosides according to claim 1, wherein the step (b) is performed at a temperature ranged from 4° C. to 90° C.

17. The process of extracting glycosides according to claim 16, wherein the step (b) is performed at the temperature ranged from room temperature to 70° C.

18. A process of extracting glycosides according to claim 1, wherein the step (b) is carried out in water bath.

19. The process of extracting glycosides according to claim 1, wherein the step (b) further comprises the step of adding an alcohol to the extractive solution.

20. The process of extracting glycosides according to claim 19, wherein the alcohol is one of C₁-C₄ alcohols with a concentration ranged from 0.5 wt %-30 wt %.

21. The process of extracting glycosides according to claim 20, wherein the alcohol is ethanol.

22. The process of extracting glycosides according to claim 1, wherein the step (c) further comprises the step of centrifugation in order to remove the aqueous layer.

23. The process of extracting glycosides according to claim 1, wherein in step (c), the aqueous layer is concentrated in vacuo.

24. The process of extracting glycosides according to claim 1, wherein the concentration of the solvent in step (d) used to wash the solid is approximately 60%-99%.

25. The process of extracting glycosides according to claim 24, wherein the solvent is an alcohol.

26. The process of extracting glycosides according to claim 25, wherein the solvent is one of C₁-C₄ alcohols.

27. The process of extracting glycosides according to claim 26, wherein the solvent is ethanol.

28. The process of extracting glycosides according to claim 1, wherein after the solid is washed, the step (d) further comprises the steps of
(d1) sonicating; and
(d2) centrifugating to separate a first filtrate and a first residue; and
wherein in the step (e) the first filtrate is concentrated to obtain the final product rich in glycosides.

29. The process of extracting glycosides according to claim 28, after the step (d2), further comprising the steps of:
(d3) sonicating; and
(d4) centrifugating to separate the solid and liquid, and forming a second filtrate and a second residue.
wherein in the step (e) the first filtrate and the second filtrate are concentrated to obtain the final product rich in glycosides.

30. The process of extracting glycosides according to claim 1, wherein in step (c), the aqueous layer is concentrated in vacuo.

31. A process of extracting glycosides from a herb, comprising the steps of:
(a) grinding the herb, mixing with water thoroughly, and agitating to form a first crude extract and a first herbal dregs,
adding water to the first herbal dreg, mixing and agitating to form a second crude extract and a second herbal dregs, and
collecting the first crude extract and the second crude extract to form an extractive solution;
(b) concentrating the extractive solution to form an extractive solution concentrate with a solid content of 1-10%;
(c) adding about 5 wt %˜30 wt % of salt and about 5 wt %˜30 wt % of polyol to the extractive solution concentrate and then mixing thoroughly, and performing a phase separation procedure at a temperature of about 4˜90° C. to form an aqueous layer and a polyol layer;
(d) removing the aqueous layer after centrifugation, and concentrating the aqueous layer in vacuo to a solid;
(e) washing the solid with ethanol in the concentration of about 60%˜99%, followed by sonicating, centrifugating, and filtration to obtain a first filtrate and a first residue.
(f) concentrating the first filtrate to obtain a final product rich in glycosides.

32. The process of extracting glycosides according to claim 31, wherein the herb is root of herbaceous peony.

33. The process of extracting glycosides according to claim 31, wherein the herb is dried gardenia fruit.

34. The process of extracting glycosides according to claim 31, wherein the herb is rhubarb.

35. The process of extracting glycosides according to claim 31, wherein the glycosides include monosaccharide, disacchrides or polysacchride.

36. The process of extracting glycosides according to claim 35, wherein the glycosides consist of sennoside, geniposide, paeoniflorin, glycyrrhizin, guercitrin, puerarin, hesperidin, ginsenoside Rbl and naringin..

37. The process of extracting glycosides according to claim 31, wherein the salt used in step (c) is selected from the group consisting of dihydrogen phosphate, hydrogen phosphate and phosphate.

38. The process of extracting glycosides according to claim 31, wherein the salt in step (c) is sulfate.

39. The process of extracting glycosides according to claim 31, wherein the salt in step (c) is chloride.

40. The process of extracting glycosides according to claim 31, wherein the salt in step (c) is oxalate.

41. The process of extracting glycosides according to claim 31, wherein the salt in step (c) is acetate.

42. The process of extracting glycosides according to claim 31, wherein the step (e) further comprises the step of washing the solid for several times with ethanol with a concentration of about 60%-99%, then sonicating, centrifugating, and filtrating to form the second filtrate and the second residual, and wherein in the step (f) the first filtrate and the second filtrate are mixed and concentrated to obtain the final product rich in glycosides.

43. An aqueous two-phases system for extracting glycosides from a plant comprising:
an extractive solution;
about 5 wt %-30 wt % of salt; and
about 5 wt %-30 wt % of polyol,
wherein the weight percentage of the salt and the polyol are calculated based on the weight of the extractive solution.

44. The aqueous two-phases system according to claim 43, wherein the salt is selected from the group consisting of dihydrogen phosphate, hydrogen phosphate and phosphate.

45. The aqueous two-phases system according to claim 43, wherein the salt is sulfate.

46. The aqueous two-phases system according to claim 43, wherein the salt is chloride.

47. The aqueous two-phases system according to claim 43, wherein the salt is oxalate.

48. The aqueous two-phases system according to claim 43, wherein the salt is acetate.

49. The aqueous two-phases system according to claim 43, further comprising alcohol.

50. The aqueous two-phases system according to claim 49, wherein the alcohol is one of C₁-C₄ alcohols.

51. The aqueous two-phases system according to claim 50, wherein the alcohol is ethanol.

52. The aqueous two-phases system according to claim 49, wherein the concentration of ethanol is in the range of 0.5 wt %-30 wt % approximately.
